(19) United States
(12) Patent Application Publication
(10) Pub. No.: US 2007/0142852 A1
(43) Pub. Date: Jun. 21, 2007

(54) Tissue cutting device

(75) Inventors: Roberta Lee, Redwood City, CA (US); Huddee Jacob Ho, San Jose, CA (US); Samuel E. Zuckswert, Sacramento, CA (US)

Correspondence Address: Jung-hua Kuo;Attorney At Law, PO Box 3275, Los Altos, CA, 94024, US

(73) Assignee: MANOA MEDICAL, INC., A DELAWARE CORPORATION, Redwood City, CA (US)

(21) Appl. No.: 11/316,600

(22) Filed: Dec. 21, 2005

(51) Int. Cl. A61B 17/32

(52) U.S. Cl. 606/170

(57) ABSTRACT

Devices and methods for efficient severing or cutting of a material or substance, such as soft tissue, suitable for use in open surgical and/or minimally invasive procedures, such as percutaneous procedures in breast tissue, are disclosed. A tissue cutting device may generally include a probe, a cutting assembly configured to be in a storage configuration or a preformed cutting configuration for cutting the specimen, a tissue fixator and a specimen retriever. When in the cutting configuration, the cutting assembly may be configured to move and cut the specimen relative to the tissue fixator along an axis of the probe. The tissue fixator may facilitate in stabilizing a region of tissue during cutting of the specimen. The region of tissue may be the specimen and/or tissue adjacent to and/or near the specimen. The specimen retriever may optionally be coupled to the cutting assembly and integrated as part of the tissue fixator.

### CROSS REFERENCE TO RELATED APPLICATIONS

This application is related to co-pending U.S. patent application Ser. No. 10/815,912 (Attorney Docket No. MNOAP008), entitled “Tissue Cutting Devices and Methods” and filed on Mar. 31, 2004, the entirety of which is incorporated by reference herein.

### BACKGROUND OF THE INVENTION

1. Field of the Invention

The present invention relates generally to devices for cutting a material or substance. More specifically, devices and methods for efficient severing or cutting of a material or substance, such as soft tissue, suitable for use in open surgical and/or minimally invasive procedures, such as percutaneous procedures in breast tissue, are disclosed.

2. Description of Related Art

Standard methods of severing of tissue may include using a scalpel, scissors, and radio frequency energy. Percutaneous procedures in soft tissue such as the breast, however, are difficult to perform using a standard scissors and scalpel as there is no exposed cavity or space as in open surgical procedures. There is continuous pressure or force of adjacent tissue on the cutting device which may affect or impede the operation of the cutting device. Furthermore, in a closed environment, radio frequency current, a common type of energy used to sever tissue, dissipates into the surrounding tissue decreasing the ability to achieve a current of sufficient high density at the cutting electrode to initiate a cut. To overcome this problem, high power settings are often required to initiate the cut which is often painful and increases thermal damage to the tissue.

In a closed environment, it may be difficult for deformable cutting mechanisms to achieve a desired configuration. Often during insertion of a percutaneous device into tissue, the cutting mechanism is housed within a probe or sheath to facilitate insertion. When the cutting mechanism is exposed for example, by advancement out of the probe or retraction of the sheath, the cutting mechanism is still surrounded by the soft tissue. The soft tissue may produce sufficient pressure on the cutting mechanism to prevent the cutting mechanism from attaining a desired shape or configuration. In particular, expandable cutting loops may not fully expand, thereby impeding efficiency of cutting.

A further disadvantage of percutaneous procedures is difficulty in stabilizing tissue during the procedure. Tissue stabilization facilitates cutting of soft tissue by preventing unexpected movement(s) especially as the soft tissue is separated from surrounding tissue. In one example, suction via a vacuum source can be used to hold and stabilize tissue within a trough while a rigid, fixed diameter, oscillating cutter advances over the trough. Only a small core of tissue is obtained with each cut. Multiple cuts are often required to obtain enough cores of tissue for diagnostic accuracy.

Accordingly, there is a need for more efficient severing or cutting of tissue that can be used during minimally invasive procedures such as percutaneous procedures in breast tissue.

### SUMMARY OF THE INVENTION

Devices and methods for efficient severing or cutting of a material or substance, such as soft tissue, suitable for use in open surgical and/or minimally invasive procedures, such as percutaneous procedures in breast tissue, are disclosed. It should be appreciated that the present invention can be implemented in numerous ways, including as a process, an apparatus, a system, a device, and a method. Several inventive embodiments of the present invention are described below.

A tissue cutting device may generally include one or more deformable cutting assemblies. The cutting assembly may be of any predetermined preformed shape that is generally altered or deformed when in a storage configuration. When in a cutting configuration, the cutting assembly preferably generally returns to the predetermined preformed shape. The cutting assembly has a cross-section that may be rectangular, square, round or any other suitable shape. The cutting assembly may have one or more cutting edges. The cutting edge may be sharpened or have a set of cutting teeth disposed along at least a portion of the cutting edge. At least part of the cutting assembly may be operatively coupled to an energy source such as radio frequency, laser, ultrasonic, heating, cooling, fluid pressure and/or mechanical oscillation and/or rotation. At least part of the cutting assembly may be at least partially insulated.

The cutting assembly may be a cutting loop forming a partial or complete loop. The cutting loop may be circular, oval, square or any other suitable shape, regular or irregular. With multiple cutting loops, one cutting loop may be nested within another cutting loop. For example, a cutting assembly may be configured with a first cutting loop opposing a second cutting loop so that a first set of cutting teeth is aligned with and configured to cooperate with a second set of cutting teeth. One or more of the cutting loops may oscillate and/or rotate.

A tissue cutting device generally includes a probe defining a probe axis and the cutting assembly in a storage configuration or a cutting configuration. The cutting assembly may be at least partially retracted within the probe in the storage configuration and return to the cutting configuration when at least partially extended through one or more openings at or near a distal end of the probe. The probe may include a sheath or cover slidable between a proximal position in which the cutting assembly is at least partially in the cutting configuration and a distal position in which the sheath at least partially houses the cutting assembly in the storage configuration.

In one embodiment, when the cutting assembly returns to the cutting configuration, the cutting assembly can be initially in general alignment with the probe axis and configured to pivot relative to the probe axis about a cutting assembly pivot.

A coagulator may be incorporated into the cutting assembly to facilitate control of bleeding. For example, the coagulator may be disposed on an outer surface of each cutting blade. The coagulator can be coupled to an energy source such as a radio frequency energy, laser, cold, ultrasonic heating, and/or electrical resistive heating source.

A tissue fixator may be incorporated into the tissue cutting device. The tissue fixator may stabilize a region of tissue as it is being cut to facilitate the cutting procedure. The region of tissue may be tissue to be severed and/or tissue adjacent and/or near the tissue to be severed. The tissue fixator may grasp, penetrate or adhere to the region of tissue. For example, as a penetrator, the tissue fixator may be one or more wires that embed into the tissue to be severed. The tissue cutting device may include a base that houses the tissue fixator. The cutting assembly may be movable relative to the base and/or tissue fixator.

A specimen retriever may be incorporated into the cutting assembly and/or the probe. For example, the specimen retriever may be a deformable material that is at least partially attached to the cutting assembly and at least partially encompasses the specimen as the tissue is cut.

An internal retractor may be incorporated into the tissue cutting device. For example, the internal retractor may be disposed around the cutting assembly. When the cutting assembly is exposed to the tissue, for example, by retraction of the sheath and/or by advancement out the distal end of the probe, the cutting assembly may not substantially or fully reconfigure to the desired preformed shape due to pressure from the surrounding soft tissue. The internal retractor may push or retract the soft tissue away from the cutting assembly, facilitating the reconfiguration of the cutting assembly to the desired preformed shape. Where the device is energized using radio frequency, the internal retractor may push or retract the soft tissue away from a cutting electrode to minimize or block the dissipation of current into the soft tissue, thereby facilitating the attainment of sufficient current density on the cutting electrode to initiate the cutting process.

A method for cutting tissue generally includes positioning a distal region of a probe of a tissue cutting device adjacent to or into a region of tissue to be severed, the probe defining a probe axis, generally returning a cutting assembly to a cutting configuration from a storage configuration, activating a specimen fixator and activating the cutting assembly and specimen retriever such that the tissue cutting device severs and collects tissue. Optionally, an internal retractor may be activated prior to activating the cutting assembly.

These and other features and advantages of the present invention will be presented in more detail in the following detailed description and the accompanying figures which illustrate by way of example principles of the invention.

### BRIEF DESCRIPTION OF THE DRAWINGS

The present invention will be readily understood by the following detailed description in conjunction with the accompanying drawings, wherein like reference numerals designate like structural elements.

FIG. 1A is a perspective view and FIG. 1C is a partial top view of an exemplary embodiment of a tissue cutting device with a cutting assembly in a storage configuration.

FIG. 1B is a partial perspective view and FIG. 1D is a partial top view of the tissue cutting device of FIGS. 1A and 1C with the cutting assembly in a cutting configuration.

FIG. 2 is a perspective view of the tissue cutting device of FIGS. 1A-1D illustrating an activated tissue fixator.

FIG. 3A is a partial perspective view and FIG. 3C is a partial top view of an exemplary embodiment of a tissue cutting device having an internal retractor and with a sheath in an open position.

FIG. 3B is partial top view of the tissue cutting device of FIGS. 3A and 3C with the sheath in a closed position.

FIGS. 4A-F are partial perspective sectional views of a method for fixating, severing and removing a tissue specimen from a breast using an embodiment of the tissue cutting device.

### DESCRIPTION OF SPECIFIC EMBODIMENTS

Devices and methods for efficient severing or cutting of a material or substance, such as soft tissue, suitable for use in open surgical and/or minimally invasive procedures, such as percutaneous procedures in breast tissue, are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the spirit and scope of the invention. Thus, the present invention is to be accorded the widest scope encompassing numerous alternatives, modifications and equivalents consistent with the principles and features disclosed herein. For purpose of clarity, details relating to technical material that is known in the technical fields related to the invention have not been described in detail so as not to unnecessarily obscure the present invention.

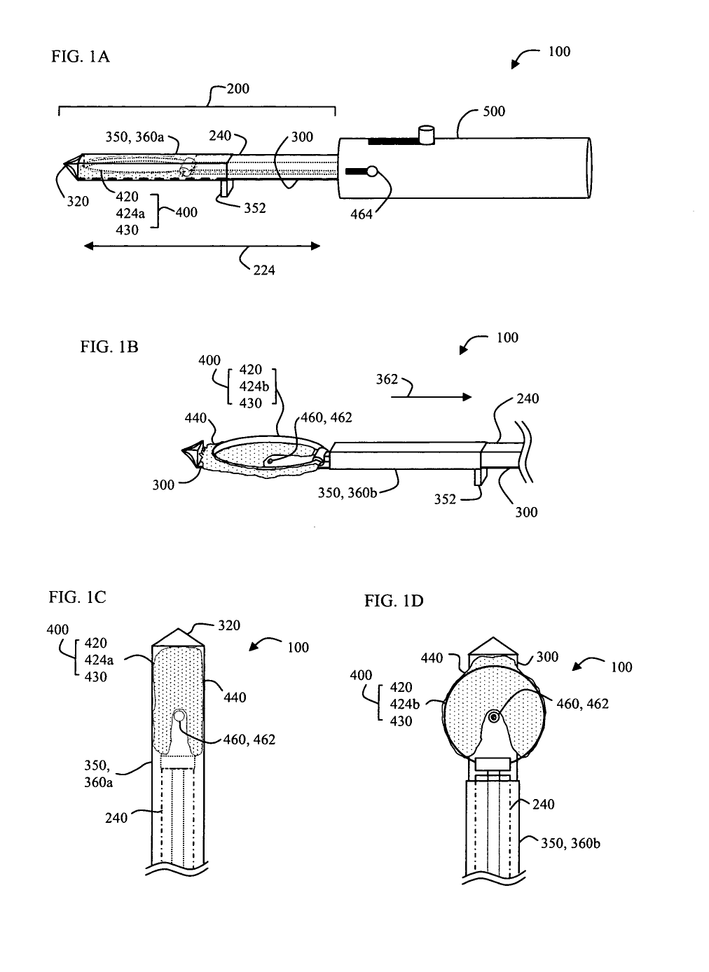

FIGS. 1A-1D illustrate an exemplary embodiment of a tissue cutting device 100 generally including a probe 200 extending from a handle 500. The probe 200 has a length that defines a probe axis 224. The probe 200 may include an inner probe 240, a base 300, and a sheath 350. A cutting assembly 400 is included in the inner probe 240.

The base 300 may be positioned on at least one side of the inner probe 240. The base 300 has a length that generally aligns with the probe axis 224. A distal end of the base 300 may include a base tip 320. The base tip 320 may optionally be sharpened to facilitate insertion into tissue. The base tip 320 may be operatively connected to an external energy source (not shown) such as radio frequency, laser, cooling, heating, ultrasonic, fluid (e.g., liquid and/or gas) pressure to facilitate insertion and positioning in soft tissue. The inner probe 240 is slidable along the length of the base 300, e.g., along the probe axis 224.

The sheath 350 can also be slidable along the length of the inner probe 240, e.g., along the probe axis 224, and the length of the base 300. As shown in FIGS. 1A and 1B, a position of the sheath 350 can be controlled by manually retracting or advancing a sheath controller 352 preferably located on the sheath 350. The sheath 350 may provide a closed position or configuration 360a as shown in FIG. 1A, in which the sheath 350 houses at least portions of the inner probe 240, the base 300, and/or the cutting assembly 400. Preferably, the base tip 320 remains exposed when the sheath 350 is in the closed position 360a. Alternatively, the sheath 350 may slide along but does not house the base 300. The closed position 360a of the sheath 350 facilitates insertion and positioning of the probe 200 into soft tissue such as breast tissue by providing a generally smooth surface, e.g., by reducing friction between the probe 200 and the tissue. Approximation of the sheath controller 352 towards the handle 500 in a direction 362, e.g., proximally, slides the sheath 350 to the open position 360b as shown in FIG. 1B to expose the cutting assembly 400.

As shown in FIG. 1B, the cutting assembly 400 may be configured as a cutting loop 420. The cutting assembly 400 is preferably deformable and may be formed of a metal, a metal alloy, ceramic, glass, plastic, a polymer, and/or any suitable combination thereof, for example. The cutting assembly 400 may be made of a material that has shape memory properties and/or superelastic properties such as a nickel titanium alloy (e.g., NiTi or nitinol), and/or a material of sufficiently high elasticity. Preferably the cutting assembly 400 is preformed to a cutting configuration 424b as shown in FIGS. 1B and 1D, as is known in the art. The cutting configuration 424b defines at least part of a circle, oval, triangle, square, rectangle, polygon, spiral or any other suitable shape that preferably optimizes the cutting of soft tissue in general or for a specific procedure depending on the application of the tissue cutting device 100.

Upon application of one or more external stresses, for example, by sliding the sheath 350 around the cutting assembly 400, the elastic and/or superelastic property of the cutting assembly 400 allows the cutting assembly 400 to configure to a storage configuration 424a, generally without the development of a permanent deformity as long as the resulting strains do not exceed the recoverable strain limits of the material of the cutting assembly 400. When the external stress(es) is removed, the cutting assembly 400 preferably returns generally to the cutting configuration 424b. For example, as shown in FIGS. 1A and 1C, the cutting assembly 400 (shown as the cutting loop 420) may be configured to be housed and stored in the storage configuration 424a within the sheath 350 when the sheath 350 is in the closed position 360a. In particular, the internal walls of the sheath 350 apply sufficient external stress to cause the cutting loop 420 to configure to the storage configuration 424a. When the cutting loop 420 is in the storage configuration 424a and housed within the sheath 350, the profile of the probe 200 is generally smaller than when the cutting loop 420 is in the cutting configuration 424b as shown in FIGS. 1B and 1D. The smaller profile of the probe 200 facilitates positioning of the probe 200 within the tissue and allows for a smaller skin incision. When generally in the cutting configuration 424b, the cutting assembly 400 may be configured to pivot around a cutting assembly pivot (not shown) relative to the probe axis.

In another alternative embodiment (not shown), the cutting assembly 400 may be advanced and/or retracted through one or more openings at or near a distal end of the probe 200. When retracted, the cutting assembly 400 may be housed within the confines of the probe 200 and is in the storage configuration 424a. When advanced through the one or more openings at or near the distal end of the probe 200, the cutting assembly 400 generally returns to the cutting configuration 424b.

The cross-sectional area (not shown) of the cutting assembly 400 may define at least part of a circle, oval, diamond, triangle, rectangle, square, any other polygon and/or any suitable combination of various shapes. The cutting assembly 400 may be energized using radio frequency, laser, ultrasound, heat, cold, oscillation, vibration, rotation, fluid pressure. The cutting assembly 400 may be operatively coupled to an external energy source (not shown). Alternatively, the energy source may be housed within the handle 500. When the cutting assembly 400 is energized by radio frequency energy, the cutting assembly 400 may be configured as a monopolar or a bipolar electrode. Activating or energizing the cutting assembly 400 may be controlled by a cutting controller (not shown) which may be located, for example, on the handle 500 or as a foot control.

The cutting assembly 400 may include one or more additional material(s) (not shown). The additional material(s) may be configured as one or more layers, portions, or segments that are continuous or non-continuous, symmetric or non-symmetric, on the surface and/or within the cutting assembly 400. The additional material(s) may provide properties such as electrical insulation, heat insulation, varying conductivity (e.g., heat and/or electrical), strength, lubricity, and/or sensors (e.g., temperature). The additional material(s) may include ceramics, polymers, plastics, metals, metal alloys, glass, diamonds, diamond-like carbon, diamond-like non-composite coating (metal-doped or nonmetal-doped) and/or various other suitable substances. One or more liquid materials may also be incorporated into the cutting assembly 400 to provide, for example, lubricity and/or heat insulation. Such materials may include, for example, silicone and perfluorinated fluids. Preferably, when radio frequency energy is used as the external energy source, the cutting assembly 400 is at least partially covered with one or more insulating materials to concentrate the cutting current on one or more edges. The insulating material is preferably of sufficient dielectric strength to prevent or reduce dissipation of the cutting current into the tissue and to concentrate the cutting current at one or more edges. Each of the one or more insulating materials is also preferably able to withstand high temperatures potentially generated by the radio frequency energy. The cutting assembly 400 may be formed using techniques and methods known in the art and may include machining, lasering, stamping, and/or chemical etching.

Referring again to FIGS. 1A-1D, the cutting loop 420 may be configured as one or more cutting blades 430 each having one or more edges to facilitate separating and/or severing the tissue. Each edge may be pointed, flat, rounded, dull, sharpened and/or serrated. Where the edge is serrated, the serrations may be continuous, intermittent, regular and/or irregular. The one or more edges may be formed using various methods such as chemical etching, machining and/or lasering. The distance between the one or more edges defines a blade separation width (not shown) which may be constant or variable along a length of the cutting blades 430. One or more of the cutting blades 430 may rotate and/or oscillate. The frequency of oscillation is preferably between 50 and 100 Hz but can also be less than 50 Hz or greater than 100 Hz. Preferably, where multiple cutting blades 430 oscillate and/or rotate, the multiple cutting blades 430 may oscillate and/or rotate in opposing directions. The oscillation and/or rotation may be powered by alternating or direct current, vacuum or fluid pressure. When direct current is used, one or more batteries may be located within or external to the handle 500.

When in the cutting configuration 424b, the cutting loop 420 (not shown) preferably has a diameter of approximately 1 to 3 cm but alternatively may be less than 1 cm or greater than 3 cm. When in the cutting configuration 424b, the cutting loop 420 may have a fixed or variable diameter.

The tissue fixator 460 facilitates in stabilizing a region of tissue during the cutting procedure. Preferably, the region of tissue is the tissue to be severed and/or is the tissue adjacent to or near the tissue to be severed. The tissue fixator 460 is preferably integrated in the tissue cutting device 100, e.g., by being at least part of and/or housed in the base 300, but may alternatively be separate from the tissue cutting device 100. When the tissue fixator 460 stabilizes the tissue to be severed, the tissue fixator 460 may also facilitate in extraction or removal of a specimen (i.e. a volume of tissue that has been severed) from the soft tissue. The tissue fixator 460 may penetrate or grasp the region of tissue and may be one or more hooks, clamps, needles and/or wires of a suitable shape. Alternatively, the tissue fixator 460 may adhere to the region of tissue and preferably attaches to the region of tissue that becomes a margin or edge of the tissue to be severed. The tissue fixator 460 may adhere to the region of tissue via a vacuum connected to an internal or external vacuum source, a biocompatible adhering substance coated or layered on the tissue fixator 460, and/or the tissue fixator 460 may be cooled to a sufficiently low temperature to attach or freeze adjacent tissue thereto. The tissue fixator 460 may be integrated with a specimen retriever 440 such that a combined tissue fixator and specimen retriever mechanism achieves both tissue fixation and specimen retrieval.

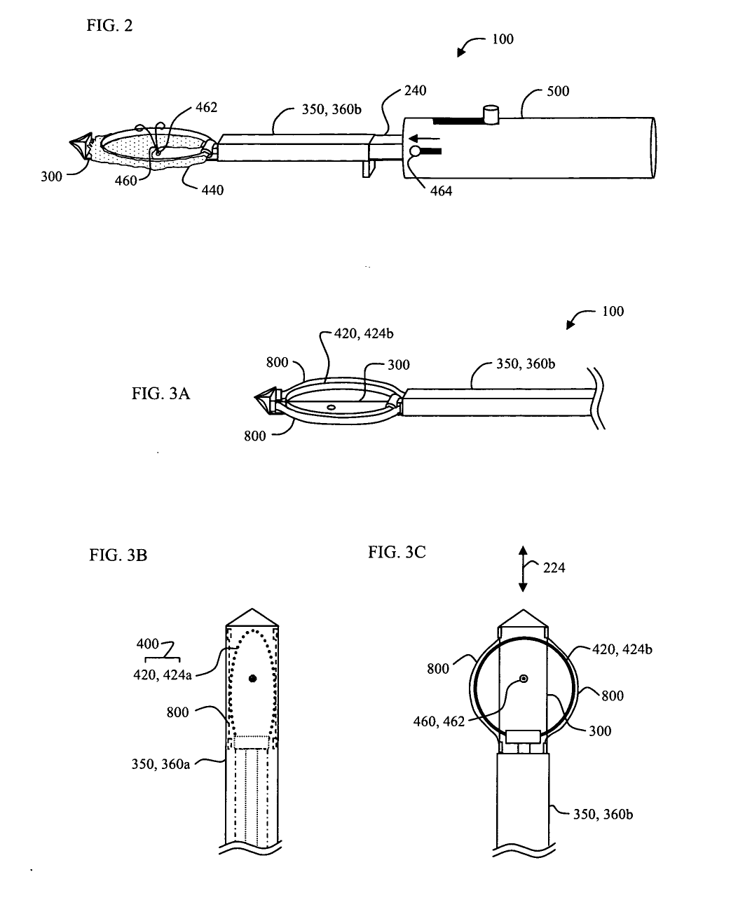

Referring again to FIG. 2, the tissue fixator 460 may be configured as a wire or needle with two times, although the tissue fixator 460 may be configured in any suitable shape or form that optimizes the fixation of tissue in general or for a specific procedure. The tissue fixator 460 is preferably formed from a material with shape memory, elastic or superelastic properties and is preferably preformed to a predetermined fixator shape. When the sheath 350 is in the closed position 360a, the tissue fixator 460 is preferably housed within a channel (not shown) in the base 300 such that the tissue fixator 460 generally conforms to the external stresses applied by the confines of the channel. When the tissue fixator 460 is advanced out of the channel through a channel opening 462, the tissue fixator 460 is released from the external stresses of the channel and generally returns to the preformed fixator shape as it penetrates into the tissue. Advancement of the tissue fixator 460 out of the channel may be controlled by manually advancing a fixator controller 464 provided, for example, on the handle 500 as shown in FIG. 1A. Preferably, the tissue fixator 460 has one or more sharpened edges and/or tips to facilitate penetration into and fixation within the tissue. Although not shown, the tissue fixator 460 may be energized using, for example, radio frequency energy to facilitate penetration into the tissue.

As shown in FIGS. 1A-D, the specimen retriever may be a deformable material or membrane that at least partially encompasses the specimen as the tissue is severed. The deformable material or membrane may be formed from a plastic, polymer, a metal, metal alloy or any deformable material, in any suitable composition, combination or variation. The polymer may be any single or combination of polyethylene, polypropylene, polyamide, polyimide, polyester, polyvinyl chloride, polyvinyl fluoride, and polytetrafluoroethylene. The specimen retriever 440 may be reinforced such as in regions or areas that may undergo more stress. Although not shown, the specimen retriever 440 may alternatively be an adherent, a penetrator or a grasper. As an adherent, the specimen retriever may comprise a cooled region of sufficient low temperature to freeze and adhere to the specimen, a region layered or coated with a biochemical adhering substance and a vacuum attached to a vacuum source. As a penetrator, the specimen retriever may be comprised of one or more wires, needles, hooks or the like.

Returning to FIGS. 1A-D, the specimen retriever 440 configured as a deformable material is shown attached in part to the cutting assembly 400 and also surrounds at least part of the base 300. As the cutting assembly 400 severs tissue, the specimen retriever 440 at least partially encompasses the severed tissue to facilitate retrieval of the specimen.

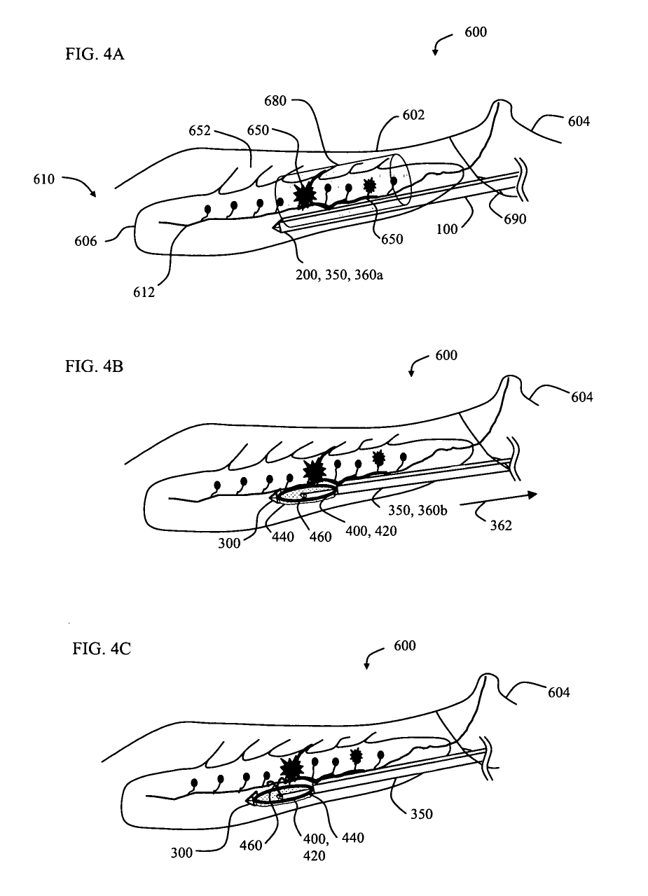

In an alternative embodiment as shown in the partial perspective view of FIG. 3A and in the partial top views of FIGS. 3B and 3C, the tissue cutting device 100 includes an internal retractor 800. FIGS. 3A and 3C illustrate the sheath 350 in the open position 360b while FIG. 3B illustrates the sheath 350 in the closed position 360a. As shown, the internal retractor 800 is preferably housed within the sheath 350. The internal retractor 800 may be activated by advancing a retractor controller (not shown) located, for example, on the handle 500 or in an alternative, the fixator controller 464 may control activation of both the tissue fixator 460 and the internal retractor 800. Activation of the internal retractor 800 expands the internal retractor 800 outward away from the probe axis 224 to facilitate in reducing or eliminating external pressure from adjacent tissue on the cutting assembly 400 by forcing or retracting tissue away from the cutting assembly 400. This in turn facilitates the return of the cutting assembly 400 generally to the preformed cutting configuration 424b. When radio frequency energy is used to energize the cutting assembly 400, the internal retractor 800 may facilitate initiation of tissue cutting by preventing or reducing the amount of current dissipation into the tissue as the cutting assembly 400 is energized and may thereby facilitate attainment of sufficient current density in the cutting assembly 400 to initiate the cutting process. In particular, by forcing tissue away from the cutting assembly 400, the internal retractor 800 helps to decrease or eliminate the amount of contact between the cutting assembly 400 and adjacent tissue and thus facilitates insulation of the cutting assembly 400 from the tissue. The internal retractor 800 may be configured to various shapes or forms and out of various materials so as to optimize the forcing of tissue away from, reduction of pressure from adjacent tissue on and/or insulation from surrounding tissue from the cutting assembly 400. In a further alternative, the internal retractor 800 may force tissue away upon inflation of, for example, a balloon.

It is noted that, although not shown, various additional components may be incorporated in the tissue cutting device 100. For example, a coagulator may be incorporated into the cutting assembly 400 to facilitate control of bleeding. The coagulator may be disposed on an outer surface of each cutting blade. The coagulator may be coupled to an energy source such as a radio frequency energy, laser, cooling, ultrasonic heating, and/or electrical resistive heating source. The coagulator may be an inductive coil configured around at least a portion of at least one of the first and second cutting blades. An energy source may be coupled to the coagulator to deliver an electrical current through the inductive coil to cause at least part of the cutting assembly 400 surrounded by the inductive coil to increase in temperature through inductive heating. A temperature sensor may also be incorporated into the cutting assembly 400 to provide a feedback mechanism for controlling a temperature of at least one of the cutting blades and the coagulator.

As a further example of an additional component, a tissue marker may be included in the cutting assembly 400. The tissue marker may be one or more dyes provided on the cutting assembly 400 and/or the tissue fixator 460. The one or more dyes may mark the specimen, preferably as the tissue is severed, to enable identification of specific sides or margins of the specimen for later orientation, for example, superficial margin, deep margin, and/or lateral margin, in relation to the breast from which the specimen was removed. As yet another example, an imaging, tracking, and/or locating device may be incorporated into the tissue cutting device 100. For example, the imaging, tracking, and/or locating device may be a light operatively connected to an internal or external source. As yet a further example, the tissue cutting device 100 may include one or more channels for evacuation of fluids and/or material from the cutting area and/or for instillation of fluid(s) and/or other substance(s) into the cutting area. The one or more channels may be operatively connected to a vacuum source and/or to a source(s) for fluid and/or other substance(s).

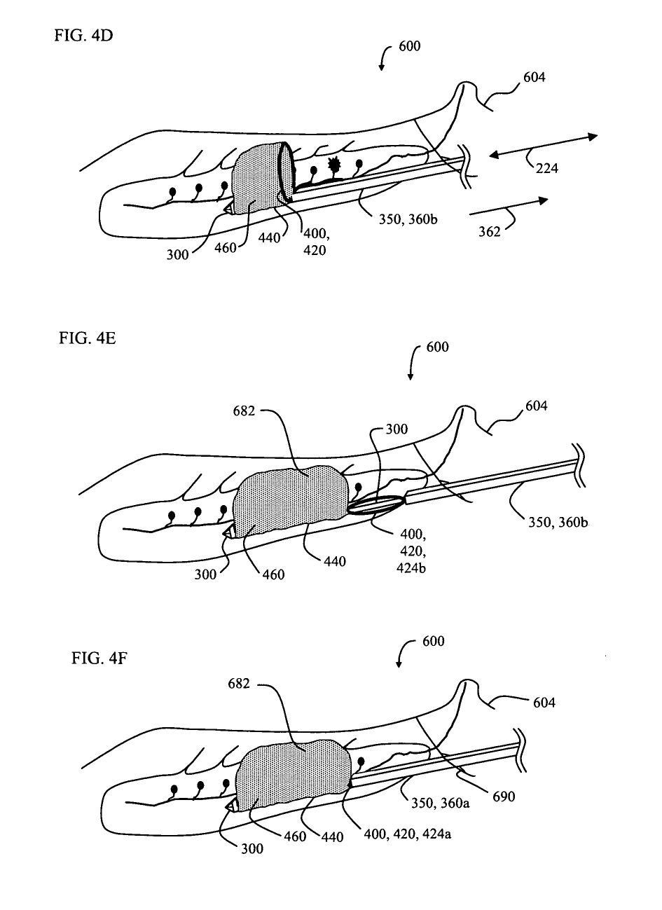

FIGS. 4A-4F are partial perspective sectional views of a method for fixating, severing and removing a tissue specimen from a breast 600 using an embodiment of the tissue cutting device 100. As shown, deep to a skin surface 602 of the breast 600 is a lobe 606 that extends from a nipple/areolar complex 604 towards a periphery 610 of the breast 600. One or more ducts, herein depicted as a main duct 612, extend generally along a length of the lobe 606. A lesion 650 is shown within part of the lobe 606. The lesion 650 may be one or more benign lesions, an invasive cancer, an extension of the cancer in the main duct 612, in duct branches (not shown) and/or in Cooper's ligament(s) and/or any multifocal cancer or cancer confined to the main duct 612. In FIG. 4A, an estimated volume of tissue 680 to be excised that contains part (e.g., biopsy) or all of the lesion 650 is shown. When the estimated volume of tissue 680 contains all of the lesion 650, preferably a margin of normal tissue surrounding the lesion 650 is included (e.g., therapeutic excision). Although the estimated volume of tissue 680 contains part of the lobe 606, the estimated volume of tissue 680 may encompass almost all of a lobe 606, an entire lobe or more than one lobe 606 and/or part of a surrounding tissue 652 of the breast 600 depending on the size and extent of the lesion 650 and the purpose of the procedure, e.g., biopsy or therapeutic excision.

The lesion 650 may be targeted using a medical targeting device (not shown). Preferably the medical targeting device is an imaging device such as a device for ultrasound imaging, magnetic resonance imaging, computerized tomography, positron emission tomography, nuclear and x-ray imaging. The imaging device may use analog and/or digital imaging technologies. The imaging device produces two-dimensional, three-dimensional and/or four-dimensional images. Preferably the imaging device images at least part of the lesion 650, the estimated volume of tissue 680 and at least a portion of the probe 200 of the tissue cutting device 100. The medical targeting device may be positioned adjacent to the skin surface 602, at a distance from the skin surface 602 and/or within the breast 600. When located in the breast 600, the medical targeting device may be attached to or incorporated in the tissue cutting device 100 or may be separate from the tissue cutting device 100. Preferably the medical targeting device is also used to guide the procedure using the tissue cutting device 100. Although not shown, one or more locators may also be positioned on the tissue cutting device 100, preferably at or near a distal end of the probe 200. The locators provide a different and/or enhanced method of identifying at least part of the probe 200 within the tissue, for example, using any suitable type of light emission. A locator sensor preferably located external to the skin may be utilized to detect and identify the position of the locator.

After the estimated volume of tissue 680 is determined, the breast 600 is prepared and local anesthetic may be administered using standard surgical technique. A skin incision 690 is made preferably using a surgical scalpel and preferably at a border of the nipple/areolar complex 604. As shown in FIG. 4A, the probe 200 is inserted through the skin incision 690 and preferably positioned under the estimated volume of tissue 680. In one embodiment (not shown), an introducer may be inserted into the breast 600 prior to insertion of the probe 200 to facilitate accurate positioning of the probe 200. The introducer may include, for example, a needle guide, a dilator and a guide sheath. The needle guide may be positioned under the estimated volume of tissue 680. After adequate positioning is determined, the dilator and guide sheath slide over the needle guide. The dilator enlarges a track around the needle guide and then the dilator and needle guide are removed, leaving the guide sheath in place. In an alternative (not shown), the introducer may include an introducer sheath. The introducer may have a sharpened tip and/or an energized tip to facilitate insertion and positioning in the breast 600. The introducer and introducer sheath are positioned in the breast 600. After adequate positioning is determined, the introducer is removed leaving the introducer sheath in place. The inner probe 240 and base 300 and/or preferably a probe cover (not shown) may be positioned at the end of the guide sheath or the introducer sheath outside of the breast 600. The inner probe 240 and base 300 may then slide through preferably the probe cover and within the guide sheath or the introducer sheath and into the breast 600 until the cutting assembly 400 is distal to the end of the guide sheath or the introducer sheath that is in the breast 600. The base 300 preferably does not have a sharpened and/or energized tip.

The process or method for fixating, severing and removing a tissue specimen from a breast 600 using the tissue cutting device 100 is now described in more detail with reference to FIGS. 4A-4F. As shown in FIG. 4A, the probe 200 is positioned adjacent to the estimated volume of tissue 680 with the sheath 350 in the closed position 360a. Preferably, the probe 200 is positioned under the estimated volume of tissue 680. The probe 200, adjacent to the estimated volume of tissue 680, provides for a first margin or edge (not shown) of a specimen 682 to be cut. As shown in FIG. 4B, the sheath 350 is retracted in the direction 362 to the open position 360b to expose the cutting assembly 400 (shown as the cutting loop 420), the specimen retriever 440, and the tissue fixator 460. As shown in FIG. 4C, the tissue fixator 460 (shown as a wire) can than be advanced out of the base 300 (such as via a channel, not shown, provided in the base 300) to penetrate into and fixates the estimated volume of tissue 680 to be severed (shown in FIG. 4A). Fixation of the estimated volume of tissue 680 facilitates the cutting procedure by stabilizing the estimated volume of tissue 680 and by providing countertraction to the movement of the cutting loop 420 in the direction 362. In an alternative (not shown), the tissue fixator 460 is preferably contained within the base 300 and may attach to the first margin of the specimen 682 to be cut and not penetrate the volume of tissue 680. The tissue fixator 460 may be cooled to a temperature sufficient to freeze and attach tissue along the first margin of the specimen 682 to be cut, a biochemical adhering substance or a vacuum attached to a vacuum source.

The cutting loop 420 may be activated to facilitate severing or cutting of the tissue and is pivoted or raised, e.g., to approximately 90° relative to the probe axis 224 as shown in FIG. 4D. In an alternative (not shown), prior to activation of the cutting loop 420, the tissue retractor may be activated to force or retract adjacent tissue away from the cutting loop 420 so as to facilitate reconfiguration of the cutting loop 420 to the cutting configuration 424b and/or to facilitate initiation of the tissue cut when using radio frequency energy.

After the cutting loop 420 is raised, the base 300 may be stabilized manually or by a mechanism (not shown) located on the tissue cutting device 100. For example, a spring positioned between the base 300 and the handle 500 may be activated to apply sufficient pressure to the base 300 in a direction opposing direction 362 so as to prevent the base 300 from moving in the direction 362 as the inner probe 240 containing the cutting loop 420 and sheath 350 are retracted in the direction 362. With the base 300 stabilized and in a relatively fixed position relative to the breast 600, the inner probe 240 and sheath 350 are retracted toward and at least partially out of the skin incision 690 to move the cutting loop 420 in direction 362, thereby creating a generally circumferential separation of the specimen 682 from the breast 600. The inner probe 240 and sheath 350 are retracted until the cutting loop 420 is generally proximal to the estimated volume of tissue 680 relative to the skin incision 690 such that when the cutting loop 420 is lowered, the cutting loop 420 is proximal to the estimated volume of tissue 680 as shown in FIG. 4E. Lowering the cutting loop 420 when it is proximal to the estimated volume of tissue 680 completes severing of the specimen 682 from the breast 600.

At the initiation of the cut as the cutting loop 420 is raised, the specimen retriever 440 configured from a deformable material and at least partially attached to the cutting loop 420, is expanded. The specimen retriever 440 generally encompasses and at least partially isolates the specimen 682 from the surrounding tissue as the cutting loop 420 is retracted. The base 300 remains adjacent to the first margin of the specimen 682. In the method herein described, the specimen retriever 440 surrounds at least part of the base 300 in addition to the specimen 682. In an alternative (not shown), the specimen retriever 440 adheres or attaches to part of the specimen 682. The specimen retriever 440 may be may be cooled to a temperature sufficient to freeze and attach to part of the specimen 682, a biochemical adhering substance or a vacuum attached to a vacuum source. In a further alternative (not shown), the tissue fixator 460 and the specimen retriever 440 are integrated.

As shown in FIG. 4F, once the severing of the specimen 682 from the breast 600 is complete, the sheath 350 may be advanced over the cutting loop 420 to the closed position 360a to facilitate removal of the probe 200 from the breast 600. The probe 200 containing the specimen 682 fixated to the tissue fixator 460 on the base 300 and at least partially within the specimen retriever 440 may then be retracted through the skin incision 690 and out of the breast 600 (not shown). As is evident, the specimen retriever 440, the base 300, and the tissue fixator 460 facilitate removal of the specimen 682.

While the exemplary embodiments of the present invention are described and illustrated herein, it will be appreciated that they are merely illustrative and that modifications can be made to these embodiments without departing from the spirit and scope of the invention. Thus, the scope of the invention is intended to be defined only in terms of the following claims as may be amended, with each claim being expressly incorporated into this Description of Specific Embodiments as an embodiment of the invention.

What is claimed is:

1. A tissue cutting device, comprising:
a probe defining a probe axis;
a tissue fixator configured to facilitate in stabilizing a region of tissue during a cutting of a specimen, the region of tissue being at least one of the specimen, tissue adjacent to the specimen, and tissue near the specimen;
a cutting assembly having a preformed cutting configuration for cutting the specimen, the cutting assembly being deformable from the cutting configuration into a storage configuration, the cutting assembly being further configured to move and cut the specimen relative to the tissue fixator along the probe axis when in the cutting configuration; and
a specimen retriever, the specimen retriever being at least one of coupled at least in part to the cutting assembly and integrated as part of the tissue fixator.

2. The tissue cutting device of claim 1, wherein the probe includes a base, the cutting assembly being slidable relative to the base.

3. The tissue cutting device of claim 2, wherein the base includes at least part of at least one of the specimen retriever and the tissue fixator.

4. The tissue cutting device of claim 2, wherein the base has a tip at a distal end thereof, the tip being configured to facilitate penetration of the probe into tissue.

5. The tissue cutting device of claim 4, wherein the tip of the base is energized.

6. The tissue cutting device of claim 1, wherein the tissue fixator is configured to at least one of penetrate and grasp the region of tissue.

7. The tissue cutting device of claim 6, wherein the tissue fixator is selected from the group consisting of one or more wires, needles, hooks and clamps.

8. The tissue cutting device of claim 6, wherein the tissue fixator comprises a material having at least one of shape memory, elastic and superelastic properties.

9. The tissue cutting device of claim 1, wherein the tissue fixator is configured to adhere to the region of tissue.

10. The tissue cutting device of claim 9, wherein the tissue fixator is one of configured to be cooled to a temperature sufficient to freeze and adhere to the region of tissue, a biochemical adhering substance, and a vacuum attached to a vacuum source.

11. The tissue cutting device of claim 1, wherein the tissue fixator is configured to fixate tissue at or near a margin of the specimen to be severed.

12. The tissue cutting device of claim 1, wherein the specimen retriever is selected from the group consisting of a deformable material, an adherent, a penetrator and a grasper.

13. The tissue cutting device of claim 12, wherein the specimen retriever is deformable and is at least partially attached to the cutting assembly so as to at least partially encompass the specimen when the specimen is being cut by the cutting assembly.

14. The tissue cutting device of claim 12, wherein the adherent is one of freezing and adhering to the region of tissue, layered or coated with a biochemical adhering substance, and a vacuum coupled to a vacuum source.

15. The tissue cutting device of claim 12, wherein the penetrator is at least one of one or more wires, needles, and hooks.

16. The tissue cutting device of claim 1, where the specimen fixator and the specimen retriever are integrated.

17. The tissue cutting device of claim 1, wherein the cutting assembly includes a cutting loop forming one of a complete loop and a partial loop.

18. The tissue cutting device of claims 17, wherein at least one of the cutting loops is operatively coupled to an energy source, the energy source being selected from the group consisting of radio frequency, laser, ultrasonic, heat, cold, fluid pressure, oscillation and rotation.

19. The tissue cutting device of claim 1, wherein the cutting assembly includes a plurality of the cutting loops, at least one of the cutting loops is nested within another of the cutting loops.

20. The tissue cutting device of claim 19, wherein at least one of the cutting loops is operatively coupled to an energy source, the energy source being selected from the group consisting of radio frequency, laser, ultrasonic, heat, cold, fluid pressure, oscillation and rotation.

21. The tissue cutting device of claim 1, wherein the cutting assembly in the cutting configuration is configured to pivot relative to the probe axis.

22. The tissue cutting device of claim 1, wherein the cutting assembly is at least partially insulated.

23. The tissue cutting device of claim 1, wherein the cutting assembly has at least one cutting edge, the cutting edge being at least one of sharpened and having a set of cutting teeth disposed along at least a portion of the cutting edge.

24. The tissue cutting device of claim 1, wherein the probe includes an inner probe and a sheath slidable along the inner probe to alternately (1) cover the cutting assembly so that the cutting assembly is in the storage configuration and (2) expose the cutting assembly so that the cutting assembly is in the cutting configuration.

25. The tissue cutting device of claim 24, wherein the sheath slides distally to cover the cutting assembly in the storage configuration and proximally to expose the cutting assembly in the cutting configuration.

26. The tissue cutting device of claim 1, wherein the probe defines one or more openings at or near a distal region thereof from which the cutting assembly extends from the storage configuration to the cutting configuration.

27. The tissue cutting device of claim 1, further comprising a tissue marker configured to mark the specimen.

28. The tissue cutting device of claim 1, further comprising an imager, tracker or locator.

29. The tissue cutting device of claim 28, wherein the imager, tracker or locator is a light.

30. A tissue cutting device, comprising:
a tissue fixator configured to facilitate in stabilizing a region of tissue during a cutting of a specimen, the region of tissue being at least one of the specimen, tissue adjacent to the specimen, and tissue near the specimen;
a cutting assembly having a preformed cutting configuration for cutting the specimen, the cutting assembly being deformable from the cutting configuration into a storage configuration;
an internal retractor disposed at least partially around the cutting assembly to facilitate one of pushing and retracting tissue away from the cutting assembly; and
a specimen retriever configured to retrieve the specimen.

31. The tissue cutting device of claim 30, wherein the internal retractor facilitates at least one of returning the cutting assembly to the first predetermined preformed shape and decreasing the dissipation of electrical current to the tissue when radio frequency energy is used to energize the cutting assembly.

32. The tissue cutting device of claim 31, wherein the cutting assembly is configured to move relative to the tissue fixator as the cutting assembly is cutting tissue.

33. A method for cutting and removing a specimen of tissue, comprising the steps of:
positioning a distal region of a probe of a tissue cutting device adjacent to or into the specimen, the probe defining a probe axis;
activating a tissue fixator of the tissue cutting device to facilitate in stabilizing a region of tissue when a cutting assembly of the tissue cutting device is cutting the specimen, the region of tissue being at least one of the specimen, tissue adjacent to the specimen, and tissue near the specimen;
returning the cutting assembly to a preformed cutting configuration from a deformed storage configuration, the preformed cutting configuration extending exterior to the probe for cutting the specimen and the deformed storage configuration being for storage of the cutting assembly generally within the probe;
activating the cutting assembly to sever the specimen;
moving the cutting assembly while the tissue fixator is activated to sever the specimen by at least one of pivoting and moving along a direction of the probe axis; and
removing the specimen using a specimen retriever.

34. The method of claim 33, wherein the positioning is guided using a medical targeting device.

35. The method of claim 34, wherein the positioning includes imaging using an imaging device as the medical targeting device.

36. The method of claim 35, wherein the imaging is one of ultrasound, mammographic, stereotactic, computer tomography, magnetic resonance, nuclear and x-ray.

37. The method of claim 35, wherein the imaging renders at least one of two-dimensional, three-dimensional and four-dimensional images.

38. The method of claim 33, wherein activating the tissue fixator includes at least one of penetrating, grasping and adhering to the specimen.

39. The method of claim 33, wherein activating the tissue fixator includes at least one of advancing one or more wires into the specimen, fastening one or more clamps onto the specimen, and advancing one or more hooks into the specimen.

40. The method of claim 33, wherein activating the tissue fixator includes adhering to the specimen, the adhering of the tissue fixator includes at least one of activating a vacuum from a vacuum source, exposing or activating a biochemical adhering substance, and cooling the tissue fixator to attach a margin of the tissue to be severed.

41. The method of claim 33, wherein returning the cutting assembly to the preformed cutting configuration from the deformed storage configuration includes at least one of advancing the cutting assembly through one or more openings at or near a distal end of the probe and sliding a sheath along an inner probe to expose the cutting assembly such that the cutting assembly reconfigures to the cutting configuration.

42. The method of claim 33, wherein the activating the cutting assembly includes at least one of oscillation, rotation, radio frequency, laser, ultrasonic, heat, cold, and fluid pressure.

43. The method of claim 33, wherein removing the specimen using the specimen retrieve includes at least one of using a deformable membrane that at least partially encompasses the specimen as the tissue is severed, grasping the specimen, penetrating the specimen with one or more wires, needles and/or hooks, and adhering to the specimen with at least one of a vacuum, a cooled specimen retriever, and a biochemical adherent.

44. The method of claim 33, wherein removing the specimen is facilitated by the tissue fixator.

45. A method for cutting and removing a specimen, comprising the steps of:
positioning a distal region of a probe of a tissue cutting device adjacent to or into the specimen, the probe defining a probe axis;
activating a tissue fixator of the tissue cutting device to facilitate in stabilizing a region of tissue when a cutting assembly of the tissue cutting device is cutting the specimen, the region of tissue being at least one of the specimen, tissue adjacent to the specimen, and tissue near the specimen;
activating a tissue retractor to force and/or retract tissue adjacent to the specimen away from the cutting assembly;
returning the cutting assembly to a preformed cutting configuration for cutting the specimen from a deformed storage configuration;
activating the cutting assembly to sever the specimen;
moving the cutting assembly relative to the tissue fixator to sever the specimen; and
removing the specimen using a specimen retriever.
